(19) United States
(12) Patent Application Publication
(10) Pub. No.: US 2021/0393116 A1
(43) Pub. Date: Dec. 23, 2021

(54) ENDOSCOPE DEVICE AND IMAGE PROCESSING METHOD

(71) Applicant: OLYMPUS CORPORATION, Tokyo (JP)

(72) Inventor: Koichiro ITO, Tokyo (JP)

(73) Assignee: OLYMPUS CORPORATION, Tokyo (JP)

(21) Appl. No.: 17/462,487

(22) Filed: Aug. 31, 2021

Related U.S. Application Data
(63) Continuation of application No. PCT/JP2019/008537, filed on Mar. 5, 2019.

(51) Int. Cl. A61B 1/00; A61B 1/05; G06T 3/40

(52) U.S. Cl. CPC A61B 1/00186; A61B 1/00009; G02B 5/201; G06T 3/4015; A61B 1/05

(57) ABSTRACT

An endoscope device includes: an image sensor that has a first color filter and a second color filter and that acquires a first image signal based on a first light transmitted through the first color filter and a second image signal based on a second light transmitted through the second color filter; and one or more processors configured to: perform color separation processing and individual-difference correction processing; and respectively allocate the first and second image signals after the processings being performed to first and second channels of a color image signal. The color separation processing is processing in which the second and first light are subtracted from the first and second image signals, respectively. The individual-difference correction processing is processing in which, for each image signals, an error is corrected based on the difference in spectral characteristics between the color filter and a reference color filter.

### CROSS-REFERENCE TO RELATED APPLICATIONS

This is a continuation of International Application PCT/JP2019/008537, with an international filing date of Mar. 5, 2019, which is hereby incorporated by reference herein in its entirety.

### TECHNICAL FIELD

The present invention relates to an endoscope device and an image processing method.

### BACKGROUND ART

In the related art, a color image sensor that includes a primary-color or complementary-color color filter array is used in an endoscope (for example, see PTL 1).

### CITATION LIST

### Patent Literature

### {PTL 1} Japanese Unexamined Patent Application, Publication No. 2013-106692

### SUMMARY OF INVENTION

One aspect of the present invention is directed to an endoscope device including: an image sensor that has a first color filter allowing first light of a first color to be transmitted therethrough and a second color filter allowing second light of a second color to be transmitted therethrough and that acquires a first image signal based on the first light transmitted through the first color filter and a second image signal based on the second light transmitted through the second color filter; a color separation correction unit that performs color separation processing and individual-difference correction processing on each of the first image signal and the second image signal; and a color conversion unit that respectively allocates the first image signal and the second image signal, on each of which the color separation processing and the individual-difference correction processing have been performed, to a first channel and a second channel of a color image signal, wherein the color separation processing is processing for subtracting a signal based on the second light from the first image signal and subtracting a signal based on the first light from the second image signal, and wherein the individual-difference correction processing is processing for correcting an error in the first image signal based on the difference between spectral characteristics of the first color filter and spectral characteristics of a predetermined first reference color filter and correcting an error in the second image signal based on the difference between spectral characteristics of the second color filter and spectral characteristics of a predetermined second reference color filter.

Another aspect of the present invention is directed to an image processing method for processing image signals acquired by an image sensor, the image sensor having a first color filter that allows first light of a first color to be transmitted therethrough and a second color filter that allows second light of a second color to be transmitted therethrough and acquiring a first image signal based on the first light transmitted through the first color filter and a second image signal based on the second light transmitted through the second color filter, the method including: a step of performing color separation processing and individual-difference correction processing on each of the first image signal and the second image signal; and a step of respectively allocating the first image signal and the second image signal, on each of which the color separation processing and the individual-difference correction processing have been performed, to a first channel and a second channel of a color image signal, wherein the color separation processing is processing for subtracting a signal based on the second light from the first image signal and subtracting a signal based on the first light from the second image signal, and wherein the individual-difference correction processing is processing for correcting an error in the first image signal based on the difference between spectral characteristics of the first color filter and spectral characteristics of a predetermined first reference color filter and correcting an error in the second image signal based on the difference between spectral characteristics of the second color filter and spectral characteristics of a predetermined second reference color filter.

### BRIEF DESCRIPTION OF DRAWINGS

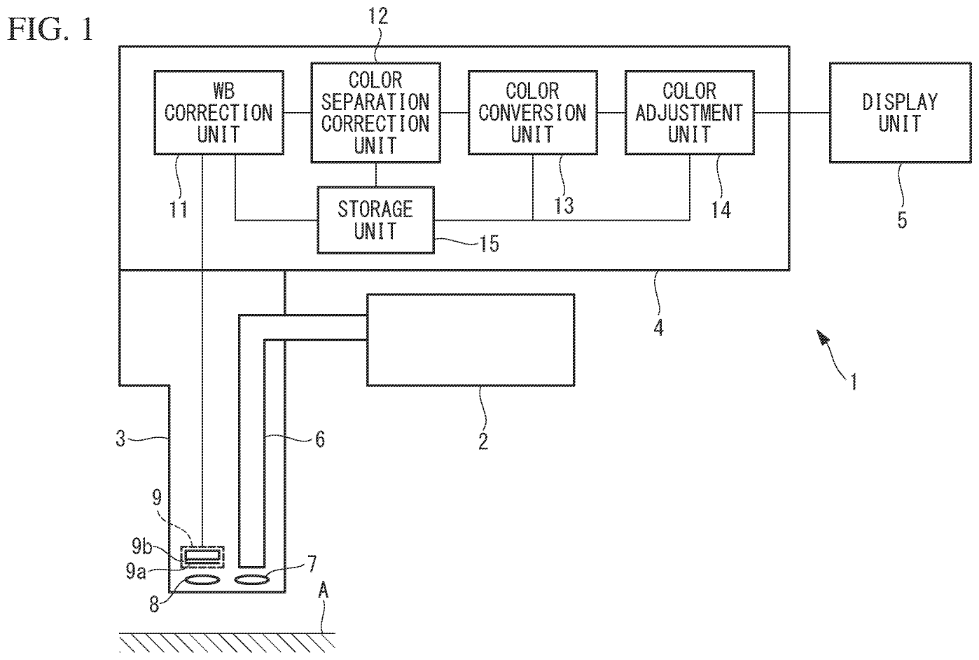

{FIG. 1} FIG. 1 is a view showing the overall configuration of an endoscope device according to one embodiment of the present invention.

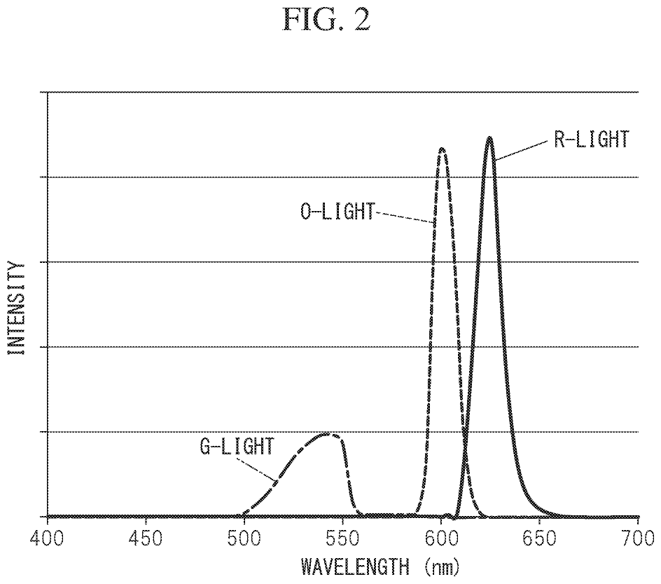

{FIG. 2} FIG. 2 is a graph showing spectral characteristics of illumination light for RBI.

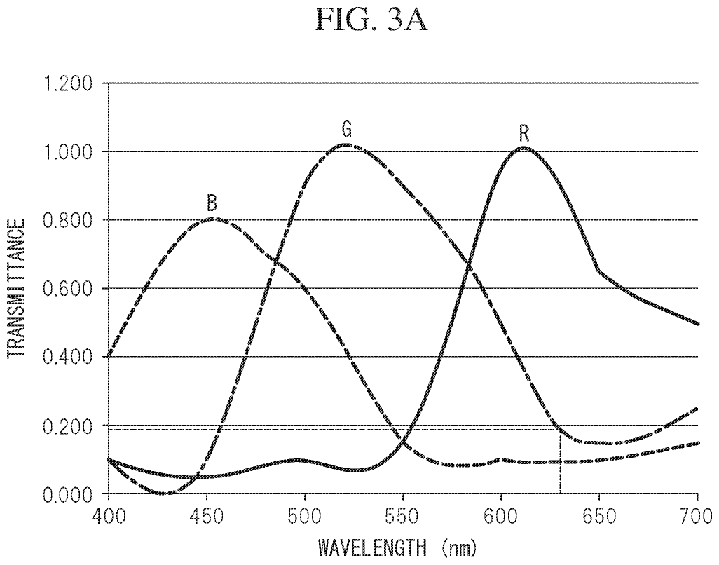

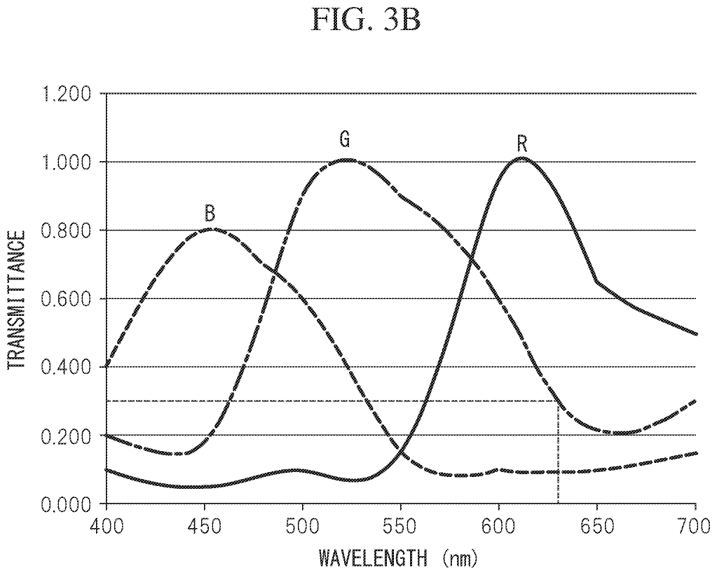

{FIG. 3A} FIG. 3A is a graph showing example spectral characteristics of an image sensor.

{FIG. 3B} FIG. 3B is a graph showing other example spectral characteristics of an image sensor.

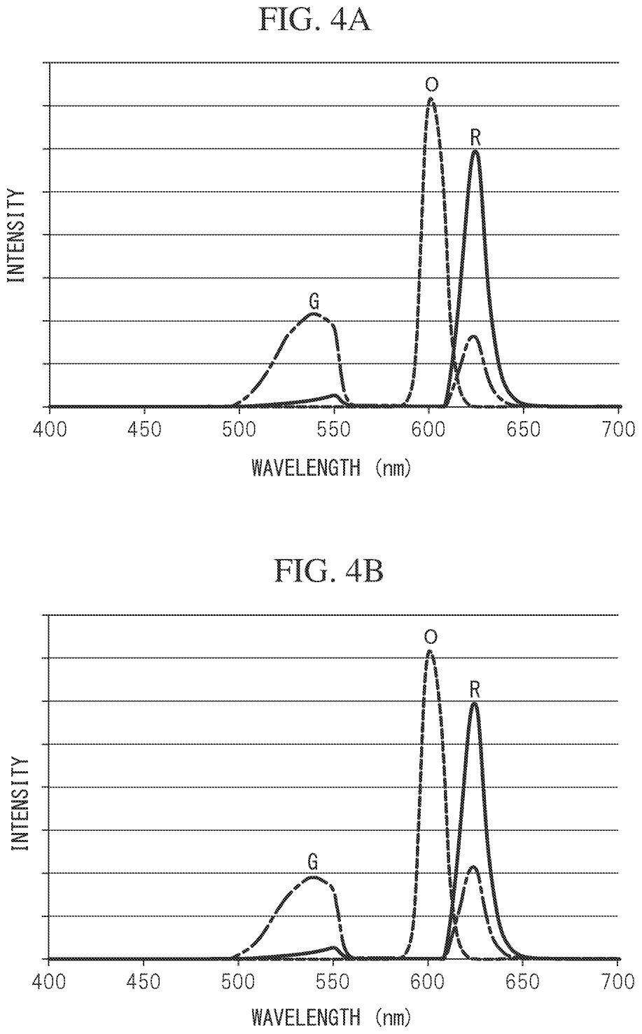

{FIG. 4A} FIG. 4A is a view showing spectral characteristics of light received at R- and G-pixels in the image sensor shown in FIG. 3A and for explaining R, O, and G image signals obtained by using the image sensor shown in FIG. 3A.

{FIG. 4B} FIG. 4B is a view showing spectral characteristics of light received at R- and G-pixels in the image sensor shown in FIG. 3B and for explaining R, O, and G image signals obtained by using the image sensor shown in FIG. 3B.

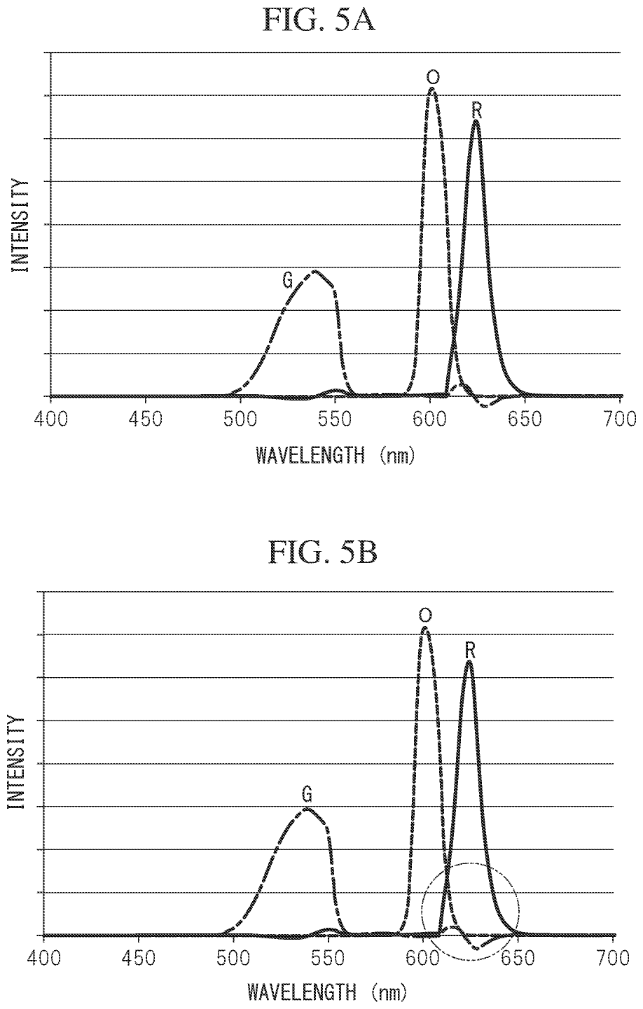

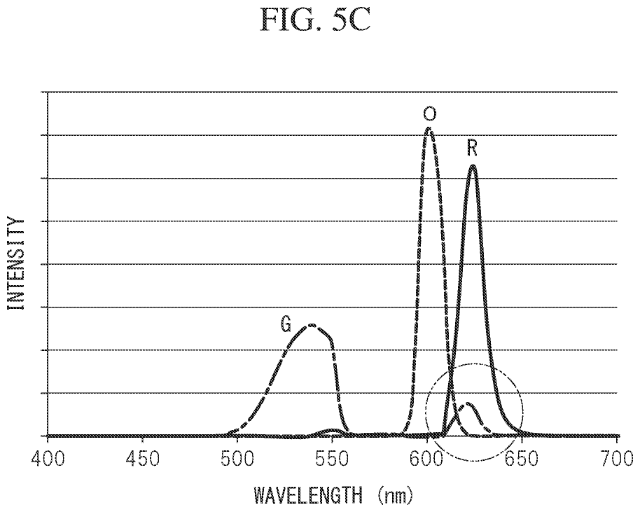

{FIG. 5A} FIG. 5A is a view for explaining the R, O, and G image signals shown in FIG. 4A obtained after color separation processing and individual-difference correction processing.

{FIG. 5B} FIG. 5B is a view for explaining the R, O, and G image signals shown in FIG. 4B obtained after the color separation processing and the individual-difference correction processing.

{FIG. 5C} FIG. 5C is a view for explaining the R, O, and G image signals shown in FIG. 4B obtained after the color separation processing.

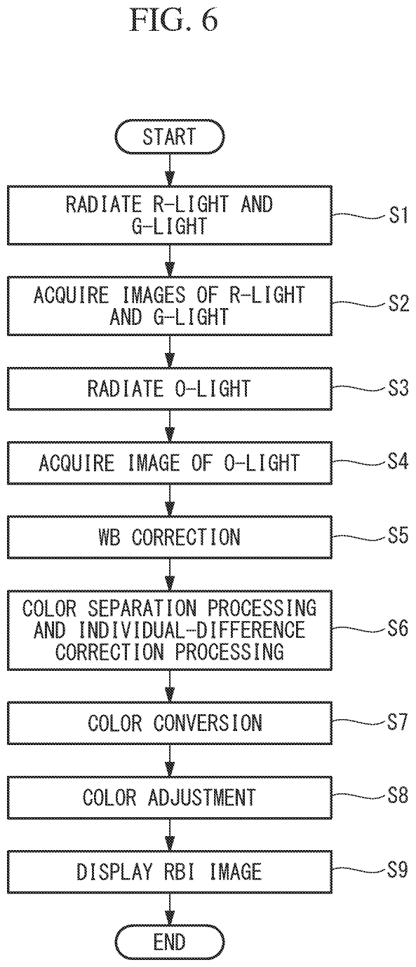

{FIG. 6} FIG. 6 is a flowchart showing the operation of the endoscope device shown in FIG. 1.

### DESCRIPTION OF EMBODIMENT

An endoscope device 1 according to one embodiment of the present invention will be described below with reference to the drawings.

As shown in FIG. 1, the endoscope device 1 of this embodiment includes: a light source device 2; an endoscope 3 that is inserted into a body; and an image processing device 4 that is connected to the endoscope 3. A display unit 5 that displays an image processed by the image processing device 4 is connected to the image processing device 4.

The endoscope device 1 has a narrow-band-light observation mode in which an RBI (Red Band Imaging) image of a subject A is observed by using red (R), orange (O), and green (G) light.

The RBI image is an image in which blood vessels in living tissue, which serve as the subject A, are emphasized. G-light reaches a surface layer of living tissue, O-light reaches a deep section below the surface layer, and R-light reaches a deeper section below the surface layer. Furthermore, the G-light, the O-light, and the R-light are absorbed by blood. Therefore, an RBI image in which blood vessels in the surface layer and the deep sections of the living tissue are clearly displayed can be obtained from the G-light, the O-light, and the R-light reflected or scattered by the subject A.

Furthermore, an RBI image is effective in identifying a bleeding point in a state in which the surface of living tissue is covered with blood bleeding from the bleeding point. Because the concentration of blood becomes higher at a bleeding point than the surroundings of the bleeding point, the permeability of O-light, in particular, is different between the bleeding point and the surroundings of the bleeding point. As a result, in an RBI image, the bleeding point and the surroundings of the bleeding point are displayed in different colors.

The endoscope device 1 further has a normal-light observation mode in which a white-light image of the subject A is observed by using white light, and may be capable of switching between the narrow-band-light observation mode and the normal-light observation mode.

The light source device 2 supplies, in the narrow-band-light observation mode, R-, O-, and G-light to an illumination optical system of the endoscope 3. FIG. 2 shows example spectral characteristics of the R-, O-, and G-light.

The R-light (second light) is narrow-band light having a peak wavelength in a wavelength band from 610 nm to 730 nm and has a peak wavelength at 630 nm, for example.

The O-light (third light) is narrow-band light having a peak wavelength in a wavelength band from 585 nm to 615 nm and has a peak wavelength at 600 nm, for example.

The G-light (first light) is narrow-band light having a peak wavelength in a wavelength band from 400 nm to 585 nm and has a peak wavelength at 540 nm, for example.

In order to generate the R-, O-, and G-light, the light source device 2 has, for example, a combination of a white light source, such as a xenon lamp, and R, O, and G color filters. Alternatively, the light source device 2 may also have three light sources (for example, LEDs or LDs) that respectively emit R-, O-, and G-light.

The light source device 2 may also supply, in the normal-light observation mode, white light to the illumination optical system.

The endoscope 3 includes: the illumination optical system, which radiates illumination light from the light source device 2 onto the subject A; and an image-acquisition optical system that receives light from the subject A to acquire an image of the subject A.

The illumination optical system has, for example, a lightguide 6 that extends from a base-end section of the endoscope 3 to a distal-end section thereof, and an illumination lens 7 that is disposed at a distal end of the endoscope 3. Light from the light source device 2 is guided from the base-end section to the distal-end section of the endoscope 3 by the lightguide 6 and is emitted from the distal end of the endoscope 3 toward the subject A by the illumination lens 7.

The image-acquisition optical system has an objective lens 8 that is disposed at the distal end of the endoscope 3 and that receives light from the subject A to form an image, and an image sensor 9 that acquires the image of the subject A formed by the objective lens 8.

The image sensor 9 is a color CCD or CMOS image sensor and has a color filter array 9a covering an imaging surface 9b. The color filter array 9a is a primary-color filter composed of R-, G-, and B-filters that are arrayed two-dimensionally. The R-, G-, and B-filters are arrayed in a Bayer array, for example, and the respective filters correspond to pixels on the imaging surface 9b. The R-filter (second color filter) transmits R-light and O-light therethrough, the G-filter (first color filter) transmits G-light therethrough, and the B-filter transmits blue light therethrough.

The image sensor 9 simultaneously images R- and G-light respectively transmitted through the R- and G-filters, and images O-light transmitted through an O-filter at a timing different from that of the R- and G-light. Therefore, the light source device 2 supplies, to the illumination optical system 6, 7, the R- and G-light and the O-light at different timings from each other. For example, the light source device 2 alternately supplies the R- and G-light and the O-light to the illumination optical system 6, 7, and the image sensor 9 alternately images the R- and G-light and the O-light. Such a synchronized operation of the light source device 2 and the image sensor 9 is controlled by, for example, a control circuit (not shown) provided in the image processing device 4. The image sensor 9 generates an R image signal (second image signal) based on the R-light, a G image signal (first image signal) based on the G-light, and an O image signal (third image signal) based on the O-light, and outputs the R image signal, the G image signal, and the O image signal to the image processing device 4.

FIGS. 3A and 3B show example spectral characteristics of image sensors 9 (spectral characteristics of R-, G-, B-filters of color filter arrays 9a). As shown in FIGS. 3A and 3B, the spectral characteristics of the image sensors 9 have variations due to individual differences in the spectral characteristics between the color filter arrays 9a. FIG. 3A shows the spectral characteristics of the average image sensor 9. Hereinafter, the average image sensor 9 having the spectral characteristics shown in FIG. 3A is referred to as a reference image sensor. FIG. 3B shows the spectral characteristics of the image sensor 9 different from the spectral characteristics shown in FIG. 3A in terms of the spectral characteristics of the G-filter. In FIG. 3B, the transmittance of the G-filter is high in the wavelength band (630 nm) of the R-light.

FIG. 4A shows spectral characteristics of light received at R- and G-pixels in the reference image sensor of FIG. 3A. FIG. 4B shows spectral characteristics of light received at R- and G-pixels in the image sensor of FIG. 3B. The R- and G-pixels correspond to the R- and G-filters, respectively. In FIGS. 4A and 4B, R-, O-, and G-spectra correspond to R, O, and G image signals, respectively.

As shown in FIGS. 4A and 4B, because the R-filter has sensitivity also in the wavelength band of the G-light, the R image signal also contains a signal based on the G-light transmitted through the R-filter. Similarly, because the G-filter has sensitivity also in the wavelength band of the B-light, the G image signal also contains a signal based on the R-light transmitted through the G-filter. Here, the amount of R-light transmitted through the G-filter in FIG. 4B is higher than that in FIG. 4A due to individual differences between the G-filters.

Note that the scale of the vertical axis is the same in FIG. 4A to FIG. 5C.

The image processing device 4 processes the R, O, and G image signals input from the image sensor 9 and generates, from one set of the R, O, and G image signals, one color image signal having three, i.e., R, G, and B, color channels.

Specifically, the image processing device 4 includes a white balance (WB) correction unit 11, a color separation correction unit 12, a color conversion unit 13, a color adjustment unit 14, and a storage unit 15.

The WB correction unit 11, the color separation correction unit 12, the color conversion unit 13, and the color adjustment unit 14 are realized by electronic circuits. Alternatively, the WB correction unit 11, the color separation correction unit 12, the color conversion unit 13, and the color adjustment unit 14 may also be realized by a processor of the image processing device 4, the processor executing processing according to an image processing program stored in the storage unit 15. The storage unit 15 has, for example, semiconductor memory such as RAM and ROM.

The R, O, and G image signals from the image sensor 9 are input to the WB correction unit 11. The storage unit 15 stores, therein, WB coefficients for the R, O, and G image signals. The WB coefficients are set on the basis of an image of a white subject A acquired by using the image sensor 9. The WB correction unit 11 multiplies the R, O, and G image signals by the corresponding WB coefficients, thereby adjusting the white balance of the R, O, and G image signals. The WB correction unit 11 outputs, to the color separation correction unit 12, the R, O, and G image signals of which the white balance has been adjusted.

The color separation correction unit 12 performs color separation processing and individual-difference correction processing on only the R and G image signals, among the R, O, and G image signals input from the WB correction unit 11. The color separation correction unit 12 outputs, to the color conversion unit 13, the R and G image signals on which the color separation processing and the individual-difference correction processing have both been performed. Meanwhile, the color separation correction unit 12 outputs, to the color conversion unit 13, the O image signal without performing any processing thereon.

In order that the color separation correction unit 12 distinguishes the R and G image signals from the O image signal, the image sensor 9 flags, for example, the R and G image signals or the O image signal. The color separation correction unit 12 determines whether to perform the color separation processing and the individual-difference correction processing on the image signals, on the basis of the presence or absence of the flag.

In the color separation processing, the color separation correction unit 12 subtracts a signal based on the G-light from the R image signal, thereby removing the signal based on the G-light from the R image signal. Similarly, the color separation correction unit 12 subtracts a signal based on the R-light from the G image signal, thereby removing the signal based on the R-light from the G image signal.

For example, an output from the R-pixel and an output from the G-pixel when the R-light is radiated and an output from the R-pixel and an output from the G-pixel when the G-light is radiated are obtained in advance. From these results, it is possible to estimate an output from the R-pixel based on the G-light (i.e., a signal based on the G-light and contained in the R image signal) and an output from the G-pixel based on the R-light (i.e., a signal based on the B-light and contained in the G image signal) that are obtained when both R-light and G-light are simultaneously radiated.

Next, in the individual-difference correction processing, the color separation correction unit 12 corrects an error in the R image signal based on the individual differences in the spectral characteristics of the R-filter, on the basis of the difference between the spectral characteristics of the R-filter and the spectral characteristics of a predetermined R reference filter (second reference color filter). Furthermore, the color separation correction unit 12 corrects an error in the G image signal based on the individual differences in the spectral characteristics of the G-filter, on the basis of the difference between the spectral characteristics of the G-filter and the spectral characteristics of a predetermined G reference filter (first reference color filter). The R reference filter and the G reference filter are, for example, an R-filter and a G-filter of the reference image sensor, which has the average spectral characteristics shown in FIG. 3A. As shown in FIGS. 5A and 5B, through the individual-difference correction processing, the R image signal is corrected so as to be approximate to an R image signal obtained when the R reference filter is used, and the G image signal is corrected so as to be approximate to a G image signal obtained when the G reference filter is used.

FIG. 5A shows the results obtained when the color separation processing and the individual-difference correction processing are performed on the R and G image signals shown in FIG. 4A. FIG. 5B shows the results obtained when the color separation processing and the individual-difference correction processing are performed on the R and G image signals shown in FIG. 4B. FIG. 5C shows, as a comparative example, the results obtained when only the color separation processing is performed on the R and G image signals shown in FIG. 4B.

For example, an R individual-difference correction coefficient and a G individual-difference correction coefficient are stored in the storage unit 15. The R individual-difference correction coefficient is set on the basis of the spectral characteristics of the R-filter of the image sensor 9 and of the R reference filter. The G individual-difference correction coefficient is set on the basis of the spectral characteristics of the G-filter of the image sensor 9 and of the G reference filter. The color separation correction unit 12 multiplies the R image signal by the R individual-difference correction coefficient and multiplies the G image signal by the G individual-difference correction coefficient.

The color conversion unit 13 generates one color image signal from the R and G image signals on which the color separation processing and the individual-difference correction processing have been performed and the O image signal. Specifically, the color conversion unit 13 allocates the R image signal to the R-channel (second channel), allocates the O image signal to the G-channel (third channel), and allocates the G image signal to the B-channel (first channel). The color conversion unit 13 outputs the color image signal, which is composed of the R, O, and G image signals, to the color adjustment unit 14.

In an example case, as shown in the following expression (1), the color separation processing and the individual-difference correction processing, which are described above, and color conversion processing are performed by using a matrix (C1, C2, . . . , C9) and a matrix (x1, x2, . . . , x9).

$$( \begin{matrix}I r \\ I g \\ I b\end{matrix} ) = ( \begin{matrix}C 1 & C 2 & C 3 \\ C 4 & C 5 & C 6 \\ C 7 & C 8 & C 9\end{matrix} ) ( \begin{matrix}x 1 & x 2 & x 3 \\ x 4 & x 5 & x 6 \\ x 7 & x 8 & x 9\end{matrix} ) ( \begin{matrix}S r \\ S o \\ S g\end{matrix} ) \qquad ( 1 )$$

The matrix (C1, C2, . . . , C9) is a matrix for the color separation processing. The matrix (x1, x2, . . . , x9) is a matrix for the individual-difference correction processing, the matrix being unique to each image sensor 9, and is determined for each image sensor 9 on the basis of, for example, inspection results after manufacturing. Sr, So, and Sg are respectively R, O, and G image signals obtained after the white-balance correction. Ir, Ig, and Ib are respectively image signals on the R-, G-, and B-channels of the color image signal.

The color adjustment unit 14 adjusts the balance of the image signals among the R-, G-, and B-channels, thereby adjusting the color of an RBI image generated from the color image signal. For example, in order to emphasize information on blood vessels in a deeper section obtained by R-light, the color adjustment unit 14 multiplies at least one of the R and G image signals by a coefficient such that the R image signal on the R-channel is increased with respect to the G image signal on the B-channel. For example, the color adjustment unit 14 multiplies a color image signal Ir, Ig, Ib by a matrix for color adjustment stored in the storage unit 15.

The image processing device 4 may also perform another processing on the image signals or the color image signal, in addition to the processing performed by the WB correction unit 11, the color separation correction unit 12, the color conversion unit 13, and the color adjustment unit 14.

Next, the operation of the thus-configured endoscope device 1 will be described below with reference to FIG. 6.

In the narrow-band-light observation mode, R-light and G-light are simultaneously supplied from the light source device 2 to the illumination optical system 6, 7 of the endoscope 3, and the R-light and the G-light are simultaneously radiated from the distal end of the endoscope 3 onto the subject A (Step S1). The R-light and the G-light reflected or scattered by the subject A are received by the objective lens 8, and an R image signal based on the R-light transmitted through the R-filter and a G image signal based on the G-light transmitted through the G-filter are simultaneously acquired by the image sensor 9 (Step S2). The R image signal and the G image signal are sent from the image sensor 9 to the image processing device 4.

Next, O-light is supplied from the light source device 2 to the illumination optical system 6, 7 of the endoscope 3, and the O-light is radiated from the distal end of the endoscope 3 onto the subject A (Step S3). The O-light reflected or scattered by the subject A is received by the objective lens 8, and an O image signal based on the O-light transmitted through the R-filter is acquired by the image sensor 9 (Step S4). The O image signal is sent from the image sensor 9 to the image processing device 4.

Steps S5 to S9, to be described below, correspond to an image processing method according to the one embodiment of the present invention.

In the image processing device 4, the white balance of the R image signal, the G image signal, and the O image signal is corrected by the WB correction unit 11 (Step S5).

Next, the color separation processing and the individual-difference correction processing are performed on the R image signal and the G image signal by the color separation correction unit 12 (Step S6). Through the color separation processing, a signal based on the G-light is removed from the R image signal, and a signal based on the R-light is removed from the G image signal. Next, through the individual-difference correction processing, an error in the R image signal based on the individual differences in the spectral characteristics of the R-filter is corrected, and an error in the G image signal based on the individual differences in the spectral characteristics of the G-filter is corrected. The R and G image signals on which the color separation processing and the individual-difference correction processing have been performed are sent to the color conversion unit 13.

The O image signal is sent to the color conversion unit 13 without being processed by the color separation correction unit 12.

Next, in the color conversion unit 13, the R, O, and G image signals are respectively allocated to the R-, G-, and B-channels of the color image signal (Step S7).

After the signal balance among the R-, G-, and B-channels of the color image signal is adjusted by the color adjustment unit 14 (Step S8), the color image signal is sent from the image processing device 4 to the display unit 5 and is displayed on the display unit 5 as an RBI image (Step S9). In the RBI image, capillary blood vessels in the surface layer are displayed in approximately yellow, blood vessels in a deep section are displayed in approximately red, and blood vessels in a deeper section are displayed in blue to black. Furthermore, blood spreading on the surface of living tissue is displayed in approximately yellow, and a bleeding point is displayed in approximately red.

For example, in the case of the image sensor 9 that has the spectral characteristics shown in FIG. 3B, because the transmittance of the G-filter in the wavelength band of the B-light is high, the G image signal contains a larger signal based on the R-light than the G image signal obtained by using the reference image sensor. Therefore, as shown in FIG. 5C, the signal based on the R-light remains, as an error, in the G image signal obtained after the color separation processing. If the image signal having such an error is used in a color image signal, the color of an RBI image becomes different from the color of an RBI image obtained by using the reference image sensor.

According to this embodiment, through the individual-difference correction processing, an error in the R image signal based on the individual differences in the spectral characteristics of the R-filter and an error in the G image signal based on the individual differences in the spectral characteristics of the G-filter are corrected, thus making it possible to obtain a color image signal equivalent to that obtained by using the reference image sensor. Therefore, color variation due to the individual differences in the spectral characteristics of the color filter array 9a is corrected, thus making it possible to generate an RBI image having colors equivalent to those obtained by using the reference image sensor.

Furthermore, according to this embodiment, the color separation processing and the individual-difference correction processing are both performed by the color separation correction unit 12. Therefore, in a case in which the color separation processing and the individual-difference correction processing are realized by circuits, it is possible to realize correction of color variation of an RBI image without complicating the circuits or increasing the sizes thereof.

In the above-described embodiment, although the color filter array 9a is a filter of the primary colors R, G, and B, instead of this, the color filter array 9a may also be a complementary-color filter composed of a Y (yellow) filter, a Cy (cyan) filter, an Mg (magenta) filter, and a G-filter.

In the above-described embodiment, although the color separation correction unit 12 performs the individual-difference correction processing after the color separation processing, instead of this, the color separation correction unit 12 may perform the color separation processing after the individual-difference correction processing. In this case, the color separation correction unit 12 subtracts a signal based on the G-light from the R image signal on which the individual-difference correction processing has been performed and subtracts a signal based on the R-light from the G image signal on which the individual-difference correction processing has been performed.

In the above-described embodiment, although the endoscope device 1 performs RBI observation in the narrow-band-light observation mode, instead of this, the endoscope device 1 may perform NBI (narrow band imaging) observation.

In this case, the light source device 2 simultaneously supplies green light (G-light) and blue light (B-light) to the illumination optical system 6, 7 of the endoscope 3. The G-light (second light) is narrow-band light having a peak wavelength in a wavelength band from 500 nm to 580 nm and has a peak wavelength at 540 nm, for example. The B-light (first light) is narrow-band light having a peak wavelength in a wavelength band from 380 nm to 460 nm and has a peak wavelength at 415 nm, for example.

The image sensor 9 generates a G image signal based on the G-light transmitted through the G-filter (second color filter) and generates a B image signal based on the B-light transmitted through the B-filter (first color filter). After the white-balance correction, the color separation processing, and the individual-difference correction processing, the G image signal is allocated to the R-channel, and the B image signal is allocated to the G-channel and the B-channel.

As a result, the above-described embodiment leads to the following aspects.

One aspect of the present invention is directed to an endoscope device including: an image sensor that has a first color filter allowing first light of a first color to be transmitted therethrough and a second color filter allowing second light of a second color to be transmitted therethrough and that acquires a first image signal based on the first light transmitted through the first color filter and a second image signal based on the second light transmitted through the second color filter; a color separation correction unit that performs color separation processing and individual-difference correction processing on each of the first image signal and the second image signal; and a color conversion unit that respectively allocates the first image signal and the second image signal, on each of which the color separation processing and the individual-difference correction processing have been performed, to a first channel and a second channel of a color image signal, wherein the color separation processing is processing for subtracting a signal based on the second light from the first image signal and subtracting a signal based on the first light from the second image signal, and wherein the individual-difference correction processing is processing for correcting an error in the first image signal based on the difference between spectral characteristics of the first color filter and spectral characteristics of a predetermined first reference color filter and correcting an error in the second image signal based on the difference between spectral characteristics of the second color filter and spectral characteristics of a predetermined second reference color filter.

According to this aspect, the image sensor acquires an image of a subject that is simultaneously irradiated with the first light and the second light, thereby simultaneously acquiring a first image signal and a second image signal. The first light and the second light are light of colors different from each other, the first image signal is generated from the first light transmitted through the first color filter, and the second image signal is generated from the second light transmitted through the second color filter. The first image signal and the second image signal are respectively allocated to a first channel and a second channel of a color image signal by the color conversion unit. From such a color image signal, it is possible to generate a color image in which an image based on the first light and an image based on the second light are superimposed.

Here, prior to allocation to the channels performed by the color conversion unit, the color separation processing and the individual-difference correction processing are performed on the first and second image signals.

The first image signal could also contain a signal based on the second light transmitted through the first color filter. Similarly, the second image signal could also contain a signal based on the first light transmitted through the second color filter. Through the color separation processing, the signal based on the second light is removed from the first image signal, and the signal based on the first light is removed from the second image signal. Therefore, in narrow-band-light observation in which narrow-band light is used as at least one of the first light and the second light, a narrow-band-light image in which specific information on the subject is emphasized can be obtained from the image signals on which the color separation processing has been performed.

Furthermore, the first image signal could also contain an error caused by the individual differences in spectral characteristics of the first color filter. Similarly, the second image signal could also contain an error caused by the individual differences in spectral characteristics of the second color filter. Through the individual-difference correction processing, it is possible to obtain the first image signal equivalent to that obtained by using the first reference color filter and the second image signal equivalent to that obtained by using the second reference color filter. Therefore, from the first and second image signals on which the individual-difference correction processing has been performed, it is possible to generate a color narrow-band-light image in which color variation caused by the individual differences in spectral characteristics of the color filters of the image sensor has been corrected.

Furthermore, because the color separation correction unit performs both color separation processing and individual-difference correction processing, in a case in which the color separation processing and the individual-difference correction processing are realized by circuits, it is possible to realize correction of color variation of an image without complicating the circuits or increasing the sizes thereof.

In the above-described one aspect, the second color filter may allow third light of a third color to be transmitted therethrough; the image sensor may acquire a third image signal based on the third light transmitted through the second color filter, at a timing different from that of the first and second image signals; and the color conversion unit may allocate the third image signal to a third channel of the color image signal.

The third light is light having a wavelength close to that of the second light. With this configuration, the subject can be observed by using the two types of light having colors close to each other.

In the above-described one aspect, the first color filter may allow the first light, which has a peak wavelength in a wavelength band from 380 nm to 460 nm, to be transmitted therethrough; and the second color filter may allow the second light, which has a peak wavelength in a wavelength band from 500 nm to 580 nm, to be transmitted therethrough.

With this configuration, NBI (Narrow Band Imaging) observation can be performed by using the first light of blue and the second light of green.

In the above-described one aspect, the first color filter may allow the first light, which has a peak wavelength in a wavelength band from 400 nm to 585 nm, to be transmitted therethrough; and the second color filter may allow the second light, which has a peak wavelength in a wavelength band from 610 nm to 730 nm, and the third light, which has a peak wavelength in a wavelength band from 585 nm to 615 nm, to be transmitted therethrough.

With this configuration, RBI (Red Band Imaging) observation can be performed by using the first light of green, the second light of red, and the third light of orange.

Another aspect of the present invention is directed to an image processing method for processing image signals acquired by an image sensor, the image sensor having a first color filter that allows first light of a first color to be transmitted therethrough and a second color filter that allows second light of a second color to be transmitted therethrough and acquiring a first image signal based on the first light transmitted through the first color filter and a second image signal based on the second light transmitted through the second color filter, the method including: a step of performing color separation processing and individual-difference correction processing on each of the first image signal and the second image signal; and a step of respectively allocating the first image signal and the second image signal, on each of which the color separation processing and the individual-difference correction processing have been performed, to a first channel and a second channel of a color image signal, wherein the color separation processing is processing for subtracting a signal based on the second light from the first image signal and subtracting a signal based on the first light from the second image signal, and wherein the individual-difference correction processing is processing for correcting an error in the first image signal based on the difference between spectral characteristics of the first color filter and spectral characteristics of a predetermined first reference color filter and correcting an error in the second image signal based on the difference between spectral characteristics of the second color filter and spectral characteristics of a predetermined second reference color filter.

According to the present invention, an advantageous effect is afforded in that it is possible to correct color variation of an image used in narrow-band-light observation, the color variation being caused by individual differences in the spectral characteristics of an image sensor.

### REFERENCE SIGNS LIST

1 endoscope device
3 endoscope
4 image processing device
9 image sensor
9a color filter array (first color filter, second color filter, third color filter)
12 color separation correction unit
13 color conversion unit

1. An endoscope device comprising:
an image sensor that has a first color filter allowing first light of a first color to be transmitted therethrough and a second color filter allowing second light of a second color to be transmitted therethrough and that acquires a first image signal based on the first light transmitted through the first color filter and a second image signal based on the second light transmitted through the second color filter; and
one or more processors comprising hardware, the one or more processors being configured to:
perform color separation processing and individual-difference correction processing on each of the first image signal and the second image signal; and
respectively allocate the first image signal and the second image signal, on each of which the color separation processing and the individual-difference correction processing have been performed, to a first channel and a second channel of a color image signal,
wherein the color separation processing is processing for subtracting a signal based on the second light from the first image signal and subtracting a signal based on the first light from the second image signal, and
wherein the individual-difference correction processing is processing for correcting an error in the first image signal based on the difference between spectral characteristics of the first color filter and spectral characteristics of a predetermined first reference color filter and correcting an error in the second image signal based on the difference between spectral characteristics of the second color filter and spectral characteristics of a predetermined second reference color filter.

2. The endoscope device according to claim 1,
wherein the second color filter allows third light of a third color to be transmitted therethrough,
wherein the image sensor acquires a third image signal based on the third light transmitted through the second color filter, at a timing different from that of the first and second image signals, and
wherein the one or more processors are further configured to allocate the third image signal to a third channel of the color image signal.

3. The endoscope device according to claim 1,
wherein the first color filter allows the first light, which has a peak wavelength in a wavelength band from 380 nm to 460 nm, to be transmitted therethrough, and
wherein the second color filter allows the second light, which has a peak wavelength in a wavelength band from 500 nm to 580 nm, to be transmitted therethrough.

4. The endoscope device according to claim 2,
wherein the first color filter allows the first light, which has a peak wavelength in a wavelength band from 400 nm to 585 nm, to be transmitted therethrough, and
wherein the second color filter allows the second light, which has a peak wavelength in a wavelength band from 610 nm to 730 nm, and the third light, which has a peak wavelength in a wavelength band from 585 nm to 615 nm, to be transmitted therethrough.

5. An image processing method for processing image signals acquired by an image sensor, the image sensor having a first color filter that allows first light of a first color to be transmitted therethrough and a second color filter that allows second light of a second color to be transmitted therethrough and acquiring a first image signal based on the first light transmitted through the first color filter and a second image signal based on the second light transmitted through the second color filter, the method comprising:
performing color separation processing and individual-difference correction processing on each of the first image signal and the second image signal; and
respectively allocating the first image signal and the second image signal, on each of which the color separation processing and the individual-difference correction processing have been performed, to a first channel and a second channel of a color image signal,
wherein the color separation processing is processing for subtracting a signal based on the second light from the first image signal and subtracting a signal based on the first light from the second image signal, and
wherein the individual-difference correction processing is processing for correcting an error in the first image signal based on the difference between spectral characteristics of the first color filter and spectral characteristics of a predetermined first reference color filter and correcting an error in the second image signal based on the difference between spectral characteristics of the second color filter and spectral characteristics of a predetermined second reference color filter.
